(19) United States
(12) Patent Application Publication
(10) Pub. No.: US 2016/0051580 A1
(43) Pub. Date: Feb. 25, 2016

(54) SUPPLY OF XENON OR ARGON GAS TO A DONOR PRIOR TO TAKING A SAMPLE OF BIOLOGICAL MATERIAL

(71) Applicant: L'AIR LIQUIDE, SOCIETE ANONYME POUR l'ETUDE ET l'EXPLOITATION DES PROCEDES GEORGES CLAUDE, Paris (FR)

(72) Inventors: Ira KATZ, Meudon (FR); Marc LEMAIRE, Paris (JP); Jan PYPE, Herne (BE)

(73) Assignee: L'AIR LIQUIDE, SOCIETE ANONYME POUR l'ETUDE ET l'EXPLOITATION DES PROCEDES GEORGES CLAUDE, Paris (FR)

(21) Appl. No.: 14/783,319

(22) PCT Filed: Apr. 9, 2014
(86) PCT No.: PCT/FR2014/050848

§ 371 Date: Oct. 8, 2015

(30) Foreign Application Priority Data
Apr. 12, 2013 (FR) 1353312

(51) Int. Cl. A61K 33/00

(52) U.S. Cl. CPC A61K 33/00

(57) ABSTRACT

The invention relates to a gaseous composition containing an active gaseous compound selected from the group made up of Xe and Ar to be used for preventing or minimising an ischaemia-reperfusion lesion of a biological material in a donor patient, while and/or immediately after taking a sample of said biological material from said donor patient, said gaseous composition being administered to the donor patient prior to taking the sample of biological material from said donor patient.

### CROSS REFERENCE TO RELATED APPLICATIONS

This application is a 371 of International PCT Application PCT/FR2014/050848 filed Apr. 9, 2014 which claims priority to French Patent Application No. 1353312 filed Apr. 12, 2013, the entire contents of which are incorporated herein by reference.

### BACKGROUND

The present invention relates to a xenon or argon gas or gas mixture intended to be administered to a donor of biological material, such as an organ or tissues, in particular a human being, prior to the removal of said organ or of said tissues so as to protect said biological material before it is extracted from the body.

The removal of biological material, i.e. of one or more organs and/or tissues, from an individual, called donor, is a surgical procedure for therapeutic purposes resulting in a transplant, i.e. the reimplantation of the biological material, called graft, in another individual, called recipient.

In order to avoid or minimize the deterioration of this biological material, i.e. of these organs and/or tissues, it is necessary to protect them with specific solutions, termed preserving solutions, or even with specific gases, with which the organs and/or tissues are brought into contact after they have been removed.

Thus, document EP-A-2536272 teaches a liquid formulation comprising a liquid solution and at least one gas chosen from xenon, argon, hydrogen, le H₂S, helium, krypton, neon, radon or CO. The gas is dissolved in the “preserving” liquid solution so as to preserve a biological material, in particular cells, tissues and biological organs, in particular an organ chosen from the heart, the kidney, the liver, the pancreas and the intestine. The gas is preferably argon.

As it happens, between the moment the removal is carried out and the moment the biological material is brought into contact with the preserving medium, deterioration of said biological material may occur, in particular ischemia-reperfusion lesions which are deleterious for the biological material.

The problem is consequently that of being able to avoid, reduce or minimize said ischemia-reperfusion lesions by protecting the biological material, in particular an organ, as soon as possible.

### DESCRIPTION OF PREFERRED EMBODIMENTS

The solution of the invention is a gaseous composition containing an active gaseous compound chosen from the group made up of Xe and Ar for use in preventing or minimizing an ischemia-reperfusion lesion of a biological material in a donor individual, during and/or subsequent to the removal of said biological material from said donor individual, said gaseous composition being administered to the donor individual prior to the removal of the biological material from said donor individual.

As appropriate, the gaseous composition of the invention may comprise one or more of the following technical characteristics:
the active gaseous compound is argon;
the active gaseous compound is xenon;
it contains one or more additional gaseous compounds chosen from N₂, H₂, He, H₂S, Kr, Ne, NO and CO;
it contains between 15% and 80% by volume of the active gaseous compound;
it contains at least 20% by volume of said active gaseous compound;
it contains at most 60% by volume of said active gaseous compound;
the biological material is a tissue, an organ or cells;
the biological material is an organ;
the biological material is an organ chosen from the liver, the kidneys, the lungs and the heart;
the active gaseous compound is mixed with an oxygen-containing gas, prior to its administration to the donor individual;
it optionally contains oxygen;
the donor individual is a human being, i.e. a man, a woman, a child, a newborn, etc.;
it is administered to a healthy donor individual;
it is administered to a donor individual whose heart is beating;
it is administered to a donor individual whose heart has stopped;
it is administered in gaseous form by inhalation;
it is in gaseous form and is administered by inhalation by means of a breathing mask or an intubation tube.

More generally, in the context of the present invention, it has been demonstrated that, in order to reduce the deterioration or lesions resulting from ischemia-reperfusions within organs or other biological tissues removed from a donor individual and intended to be reimplanted and transplanted in a recipient individual, it is advisable to act prior to the surgical extraction of said organs or biological tissues by administering to the donor a protective gaseous composition based on xenon or on argon.

In other words, a gaseous composition containing an active gaseous compound chosen from argon and xenon as a mixture with oxygen can be used in a therapeutic treatment method aimed at preventing and/or minimizing an ischemia-reperfusion lesion of a biological material in a donor individual, such as a human being, during and/or subsequent to the removal of said biological material from said donor individual, in which the gaseous composition is administered to the donor individual, preferentially by inhalation, prior to the removal of the biological material from said donor individual.

The duration of administration of the gas can be easily determined, via simple routine tests, according to the organ to be extracted and the individual under consideration.

The invention also relates to a method for removing a biological material from a donor individual whose heart has stopped, characterized in that a gaseous composition containing an active gaseous compound chosen from the group made up of xenon (Xe) and argon (Ar) is administered to said donor individual whose heart has stopped, prior to and/or during the removal of said biological material from said donor individual so as to prevent or minimize an ischemia-reperfusion lesion of said biological material.

As appropriate, the method of the invention may comprise one or more of the following technical characteristics:
the donor individual is a human being;
the biological material is an organ chosen from the liver, the kidneys, the lungs and the heart;
the gaseous composition contains at least 20% by volume of said active gaseous compound;
the gaseous composition contains between 15% and 80% by volume of said active gaseous compound;
subsequent to the removal of the biological material, said biological material is brought into contact with argon or xenon, preferably in gaseous form or in liquid form, i.e. by infusion.

### Illustrative Example

In order to show the efficacy of a gaseous composition according to the invention made up of a gaseous mixture of xenon or argon, and of oxygen for preventing or minimizing the ischemia-reperfusion lesions of a biological material in a donor individual, namely in this case the ischemia-reperfusion lesions of the lungs of pigs and of mice, the gaseous mixtures (Ar/O₂ and Xe/O₂) were administered to the anaesthetized animals (anaesthetized using propofol) by inhalation, prior to removal of their lungs.

The gaseous composition is administered to the animals by inhalation, the animals being intubated and given gas by means of a respiratory ventilator.

The compositions tested contain Ar or Xe contents of between 15% and 80% by volume, the rest being oxygen.

In this case, the administration of the gas began approximately 30 minutes before the removal of their lungs. However, this period can vary by a few minutes to several hours.

Next, a virtual removal of the lungs from the animals is carried out by clamping the lungs and the arteries and veins connected to the lungs so as to obtain total exclusion of the lungs from the body of the animals in a manner identical to an actual extraction.

Once the clamping has been carried out, a period of approximately 90 minutes is allowed to pass, and then the lungs, veins and arteries are unclamped.

A group of control animals is treated in the same manner, but with gaseous mixtures of nitrogen and oxygen (N₂/O₂) in the same proportions.

Lung ventilatory function tests are then carried out.

The animals are then sacrificed and the lungs are extracted and subjected to anatomical studies in order to determine the ischemic state, i.e. the presence of possible lesions due to the period of ischemia-reperfusion.

The results obtained show that the compositions according to the invention, based on argon or on xenon, decrease the ischemia-reperfusion lesions, and the consequences thereof on good lung function, contrary to the animals of the control group having inhaled mixtures based on nitrogen.

These tests show the value of administering a gaseous composition according to the invention to a donor individual prior to the removal of the biological material from said donor individual.

1.-15. (canceled)

16. A method of preventing or minimizing an ischemia-reperfusion lesion of a biological material in a donor individual, the method comprising the step of administering to the donor individual, prior to the removal of the biological material from said donor individual, a gaseous composition containing an active gaseous compound chosen from the group made up of xenon (Xe) and argon (Ar) to thereby prevent or minimize ischemia-reperfusion lesion of the biological material during and/or subsequent to the removal of said biological material from said donor individual.

17. The method of claim 16, wherein the gaseous composition contains between 15% and 80% by volume of said active gaseous compound.

18. The method of claim 16, wherein the biological material is a tissue, an organ or cells.

19. The method of claim 16, wherein the biological material is an organ.

20. The method of claim 16, wherein the biological material is an organ chosen from a liver, a kidney, a lung and a heart.

21. The method of claim 16, further comprising a step of mixing the active gaseous compound with an oxygen-containing gas, prior to the step of administration to the donor individual.

22. The method of claim 16, wherein the donor individual is a human being.

23. The method of claim 16, wherein the gaseous composition is administered to a healthy donor individual.

24. The method of claim 16, wherein the gaseous composition is administered to a donor individual whose heart has stopped.

25. The method of claim 16, wherein the gaseous composition is administered in gaseous form by inhalation.
